(19) United States
(12) Patent Application Publication
(10) Pub. No.: US 2010/0029771 A1
(43) Pub. Date: Feb. 4, 2010

(54) TREATMENT OF ADDICTIVE DISORDERS

(76) Inventor: Olivier Ameisen, Paris (FR)

Correspondence Address: WILSON IP LAW, 5204 DELAFIELD AVE., BRONX, NY, 10471, US

(21) Appl. No.: 12/533,608

(22) Filed: Jul. 31, 2009

Related U.S. Application Data
(60) Provisional application No. 61/085,391, filed on Jul. 31, 2008.

(51) Int. Cl. A61K 31/195; A61K 31/19; A61P 25/32; A61P 25/34; A61P 25/30

(52) U.S. Cl. 514/567; 514/557

(57) ABSTRACT

The present invention is directed to the treatment of addiction, and particularly to addictions with a chemical dependency component, with gamma-hydroxybutyrate (GHB) and at least one gamma-aminobutyric acid (B) receptor agonist (GABA_{B} receptor agonist), for example, baclofen. Pharmaceutical compositions therefor are also provided.

This application claims priority under 35 U.S.C. §119(e)(5) to U.S. Provisional Application No. 61/085,391, filed Jul. 31, 2008, which is incorporated herein in its entirety.

### FIELD OF THE INVENTION

The present invention is directed to the treatment of addiction, and particularly to addictions with a chemical dependency component, using gamma-hydroxybutyrate (GHB) and at least one gamma-aminobutyric acid (B) receptor agonist (GABA_{B} receptor agonist), for example, baclofen. Pharmaceutical compositions therefor are also provided.

### BACKGROUND OF THE INVENTION

Baclofen is known to act through presynaptic and postsynaptic pathways, and its primary site of action is the spinal cord where it reduces the release of excitatory neurotransmitters. It is used to help relax certain muscles in the body. Baclofen relieves the spasms, cramping, and tightness of muscles caused by medical problems such as multiple sclerosis, cerebral palsy, or certain injuries or diseases of the spine. It is approved for the alleviation of signs and symptoms of spasticity resulting from multiple sclerosis, for the relief of flexor spasms and concomitant pain, clonus and muscular rigidity. Baclofen has been shown to have dose-dependent suppressive properties in treating alcohol dependence (Ameisen, 2007a; Ameisen, 2005; U.S. Ser. No. 12/014,746, filed Jan. 15, 2008, published as US 2008-0182904, Jul. 31, 2008).

GHB is produced in the human body and a biological deficit of GHB may play a role in addiction and related syndromes. For example, GHB is an anti-craving agent which has been used for treating alcoholism in Italy and Austria (Nava et al., 2006).

The effects of baclofen in alcohol dependence have been described (Bucknam et al., 2007; Ameisen, 2005; Agabio et al., 2007). Clinical trials have shown baclofen to reduce anxiety in alcoholic (Krupitsky et al., 1993; Addolorato et al., 2002) and non-alcoholic subjects alike (Breslow et al., 1989; Drake et al., 2003), with somnolence being an overwhelming side effect. Baclofen and GHB have individually been shown to reduce craving in alcoholic subjects unlike other sedative/hypnotics such as benzodiazepines, meprobamate, barbiturates (Addolorato et al., 2002; Caputo et al., 2003; Nava et al., 2006). It was established that the sedative/hypnotic effect of GHB in mice was, like that of baclofen, mediated by the stimulation of GABA_{B} receptors (Carai et al., 2001). Functionally, both baclofen and GHB were seen to increase the potassium current and decrease the H-current in hippocampal neurons via GABA_{B} receptor (Schweitzer et al., 2004). Humeniuk et al. (1994) reports the effects of GABA_{B} ligands on alcohol withdrawal in mice.

In a study combining GHB and naltrexone to reduce craving in alcohol dependence, Dr. Caputo and colleagues (Caputo et al., 2007) conducted a clinical study that showed the benefits of naltrexone (NTX) on alcohol craving and intake are limited to the 3 first months of treatment (Davidson et al., 2007). The study is the first randomized trial to test a recently described model of treatment of addiction: craving-suppression (Ameisen, 2005), as verified in individual case reports (Ameisen, 2005; Bucknam et al., 2007; Agabio et al., 2007) and in an unpublished series of 12 patients (Pascal Gache, University Hospitals of Geneva, personal communication). Caputo et al. describe two cravings: one for alcohol and one for GHB. The study achieved suppression for iatrogenic craving (GHB), but only a reduction for alcohol craving (primary disease). Doing so, they give a clear illustration of the benefits of suppression over reduction. Patients on GHB monotherapy who crave GHB are in a precarious situation in which they have to deploy considerable efforts to maintain abstinence (impact on well-being) which puts them at high risk for GHB and alcohol intake and relapse (prognostic aspect). Hence, there were benefits of suppressing GHB-craving by adding NTX. By contrast, with alcohol, the Caputo study allowed persistence of craving (effort-mediated abstinence) that, as in all trials of craving-reducing medications, was accompanied by a high relapse rate (30% in their GHB/NTX combined trial). Consequently, the present inventor has discovered that baclofen's dose-dependent suppressive properties in alcohol dependence (Ameisen, 2007a), could further reduce alcohol relapse risk and improve patients' well-being, particularly in combination with GHB. Both GHB and baclofen act via the GABA_{B} receptors. Baclofen, without being tied to a mechanism, appears capable of ameliorating the GHB deficiency.

### SUMMARY OF THE INVENTION

The present invention is directed to a pharmaceutical composition comprising therapeutically effective amounts of gamma-hydroxybutyrate (GHB) and at least one GABA_{B} receptor agonist in amounts sufficient to suppress cravings associated with addiction. In a preferred embodiment, the GABA_{B} receptor agonist is baclofen. When cravings are suppressed, such suppression means that craving are gone for a lengthy period of time, for example from at least 2-4 months, to 6 months, a year or longer or are gone indefinitely.

Accordingly, the pharmaceutical compositions of the invention are useful for treating of a subject suffering from an addictive disorder, such as for example, those associated with alcohol, nicotine, cocaine, heroin, another drug, gambling, sex, bulimia, binge-eating disorders or an obsessive-compulsive disorder. The compositions of the invention contain doses of GHB and baclofen sufficient to suppress cravings associated with the addiction for which the patient is being treated.

In another aspect, the present invention provides a method of treating addiction which comprises administering GHB and a GABA_{B} receptor agonist in a single dosage form, such as the pharmaceutical composition of the invention, or in individual dosage forms, by providing dosages of each for a time and in an amount sufficient to suppress cravings associated with the relevant addiction. Such doses can and may vary with each individual and can be readily determined by those of skill in the art. One method for determining the baclofen dose is by determining the dose-dependent suppressive amounts for the subject as described in U.S. Ser. No. 12/014,746. This method is also useful for determining the GHB dose. The preferred, GABA_{B} receptor agonist is baclofen and the method is applicable for treating addiction to alcohol, nicotine, cocaine, heroin, another drug, gambling, sex, bulimia, binge-eating disorders, an obsessive-compulsive disorder, insomnia and as well as other addictions involving chemical dependencies. Additionally, the GHB and the at least one GABA_{B} receptor agonist can be administered singly (as two separate dosage forms) at once dosing period or in alternating dosing periods. Either compound may be administer first and administration may be alternated as needed.

In a further aspect, the invention provides a method to treat or ameliorate depression or other psychological condition which comprises administering GHB and a GABA_{B} receptor agonist in a single dosage form, such as the pharmaceutical composition of the invention, or in individual dosage forms, to a patient in need of treatment for a time and in an amount or amounts to relieve the associated symptoms of the depression or the condition and to provide therapeutic benefit to the patient. Such conditions, include, but are not limited to menopause, premenstrual symptoms, insomnia, obsessive-compulsive disorder, ADHD, ADD and tensions headaches.

### DETAILED DESCRIPTION

As used herein and in the claims, the singular forms “a,” “an,” and “the” include the plural reference unless the context clearly indicates otherwise. All numbers expressing quantities of ingredients or reaction conditions used herein should be understood as modified in all instances by the term “about”.

“Addictive disorder” as used herein include any kind of behavior that involves excessive craving to perform one or more actions to achieve mental relaxation to levels that are generally considered detrimental to the well-being of subject. Such addictive disorders include alcohol dependence; dependence on substances including, but not limited to cocaine, opiates such as morphine, heroin, nicotine, amphetamine and the like; binge eating; bulimia nervosa, which is generally characterized by food cravings with binge eating alone or bulimia nervosa with binge eating followed by compensatory behaviors such as but not limited to purging, vomiting, over-exercising, etc.; compulsive gambling; sex addictions; computer addictions; anxiety disorders; attention deficit disorders, and so on. Without being bound to any theory, it is hypothesized that such addictive disorders result from an adaptative phenomenon in response to an underlying GHB-deficiency related dysphoric syndrome (such as anxiety, muscular tension, insomnia, depression) in which external stimuli such as alcohol, substances including, but not limited to cocaine, morphine, heroin, and the like; binge eating; would be sought to substitute for insufficient GHB effect (Ameisen, 2007b).

Treating and controlling addictive disorders and behaviors, involves suppression of the cravings associated with the addictive disorders. Such reduction can be measured by the cessation of the relevant symptoms as well as an actual intake reduction of the substance or diminishing of the behavior being craved or acted out.

“Therapeutic amount” as used herein means the amount of drug necessary to produce a therapeutic effect. “Therapeutic effect” as used herein is the one or more effects produced by an active agent that are judged to be desirable and beneficial. For example, a therapeutically effective amount for treating alcohol addiction is that amount of the drug which suppresses the desire to drink alcohol. Similarly, for other addictions, a therapeutically effective amount is that amount of drug that suppresses the desire to engage in the addictive behavior, or to imbibe in the addictive substance, as the case may be. When such desires are suppressed, the craving for the substance or behavior is also suppressed and dependence on the substance or the behavior is alleviated. Suppression of symptoms can be assessed by self reporting to the physician and include but are not limited to the patient reporting that he/she has no cravings, no thoughts about the addicting substance or behavior, no preoccupation with the addicting substance or behavior, no dreams about the addicting substance or behavior and the like.

As used herein, the term “subject” means any mammal including humans.

“GABA_{B} receptor agonist” as used herein means a substance that binds specifically to GABA_{B} receptor and which acts to stimulate or increase the action at the GABA_{B} receptor, producing typically sedative effects. The abbreviation GABA stands for gamma-aminobutyric acid and is a neurotransmitter that acts at both GABA_{A} and GABA_{B} receptor sub-types. GABA receptors exist in the central nervous system (CNS) and the enteric nervous system.

The invention provides a pharmaceutical composition comprising a therapeutic amount of at least one GABA_{B} receptor agonist and a therapeutic amount of GHB. U.S. Pat. No. 6,664,069 provides a list of GABA_{B} receptor agonists useful in this invention. Some exemplary GABA_{B} receptor agonists include 4-aminobutanoic acid (GABA), 4-amino-3-(4-chlorophenyl)butanoic acid, 4-amino-3-phenylbutanoic acid, 4-amino-3-hydroxybutanoic acid, 4-amino-3-(4-chlorophenyl)-3-hydroxyphenylbutanoic acid, 4-amino-3-(thien-2-yl)butanoic acid, 4-amino-3-(5-chlorothien-2-yl)butanoic acid, 4-amino-3-(5-bromothien-2-yl)butanoic acid, 4-amino-3-(5-methylthien-2-yl)butanoic acid, 4-amino-3-(2-imidazolyl)butanoic acid, 4-guanidino-3-(4-chlorophenyl)butanoic acid, 3-amino-2-(4-chlorophenyl)-1-nitropropane, (3-aminopropyl)phosphonous acid, (4-aminobut-2-yl)phosphonous acid, (3-amino-2-methylpropyl)phosphonous acid, (3-aminobutyl)phosphonous acid, (3-amino-2-(4-chlorophenyl)propyl)phosphonous acid, (3-amino-2-(4-chlorophenyl)-2-hydroxypropyl)phosphonous acid, (3-amino-2-(4-fluorophenyl)propyl)phosphonous acid, (3-amino-2-phenylpropyl)phosphonous acid, (3-amino-2-hydroxypropyl)phosphonous acid, (E)-(3-aminopropen-1-yl)phosphonous acid, (3-amino-2-cyclohexylpropyl)phosphonous acid, (3-amino-2-benzylpropyl)phosphonous acid, [3-amino-2-(4-methylphenyl)propyl]phosphonous acid, [3-amino-2-(4-trifluoromethylphenyl) propyl]phosphonous acid, [3-amino-2-(4-methoxyphenyl)propyl]phosphonous acid, [3-amino-2-(4-chlorophenyl)-2-hydroxypropyl]phosphonous acid, (3-amino propyl)methylphosphinic acid, (3-amino-2-hydroxypropyl)methylphosphinic acid, (3-aminopropyl) (difluoromethyl) phosphinic acid, (4-aminobut-2-yl)methylphosphinic acid, (3-amino-1-hydroxypropyl)methylphosphinic acid, (3-amino-2-hydroxypropyl) (difluoromethyl) phosphinic acid, (E)-(3-aminopropen-1-yl)methylphosphinic acid, (3-amino-2-oxo-propyl)methyl phosphinic acid, (3-aminopropyl)hydroxymethylphosphinic acid, (5-aminopent-3-yl)methylphosphinic acid, (4-amino-1,1,1-trifluorobut-2-yl)methylphosphinic acid, (3-amino-2-(4-chlorophenyl)propyl)sulfinic acid, and 3-aminopropylsulfinic acid. In situations wherein the GABA_{B} receptor agonists are optically active, racemic mixtures and enantiomerically pure forms of the compounds are also included. Derivatives, prodrugs metabolites and any pharmaceutically acceptable salts of the aforementioned compounds are also included.

The term “analog” means a compound which comprises a chemically modified form of a specific compound or class thereof, and which maintains the pharmaceutical and/or pharmacological activities characteristic of said compound or class.

The term “derivative” means a chemically modified compound wherein the modification is considered routine by the ordinary skilled chemist, such as an ester or an amide of an acid, protecting groups, such as a benzyl group for an alcohol or thiol, and tert-butoxycarbonyl group for an amine.

The term “prodrug”, as used herein, includes any covalently bonded carriers which release an active parent drug of the present invention in vivo when such prodrug is administered to a subject. Because prodrugs are known to enhance numerous desirable qualities of pharmaceuticals (i.e., solubility, bioavailability, manufacturing, etc.) the compounds of the present invention may be made available in a prodrug form. Prodrugs of the present invention may be prepared by modifying functional groups present in the compound in such a way that the modifications are cleaved, either in routine manipulation or in vivo, to the parent compound. The transformation in vivo may be, for example, as the result of some metabolic process, such as chemical or enzymatic hydrolysis of a carboxylic, phosphoric or sulphate ester, or reduction or oxidation of a susceptible functionality.

In one specific embodiment, the GABA_{B} receptor agonist useful in the invention is 4-amino-3-(4-chlorophenyl)butanoic acid, also known as baclofen. Also included herein are racemic baclofen, enantiomerically pure L-baclofen, and analogs, derivatives, prodrugs, metabolites thereof. For example, baclofen analogs include 3-thienyl- and 3-furylaminobutyric acids. Baclofen is available from commercial sources such as Sigma Chemical Company, Milwaukee, Wis., USA, or under the trade names Lioresal™ and Kemstro™.

Also included in the pharmaceutical compositions of the invention is GHB. GHB is available from commercial sources such Sigma Chemical Company, Milwaukee, Wis., USA, or under the trade name Xyrem™ (or Alcover in Europe) and can be employed in the pharmaceutical compositions of the invention as the free acid or alcohol, or as a pharmaceutically acceptable salt or ester thereof. Pharmaceutically acceptable salts may be obtained using standard procedures well known in the art, for example by reacting a sufficiently basic compound with the carboxylic acid affording the salt. Metal or organic cations, such as Ca⁺, K⁺, Li⁺, or (R)₄N⁺ wherein each R is H, phenyl, C₁-C₆ alkyl or hydroxyl C₁-C₆ alkyl, e.g., ammonium or hydroxyethyl amine salts of the carboxylic acid can be easily made using this method. Hydroxy protecting groups such as esters, ethers, acetals and ketals may be utilized in the present compounds. Useful hydroxy protecting groups are described in Greene et al., 1991.

The pharmaceutical compositions of the invention comprising baclofen and GHB can compensate for deficient effect of the physiological GABA_{B} receptor agonist(s). Thus, the pharmaceutical composition of the invention may be used to suppress cravings and symptoms (such as obsessive thoughts, preoccupation, and the like) associated with the addictive disorder. The pharmaceutical compositions of the inventions provide tremendous advantage in that their appropriate use provides substantial relief from the addictive disorder without substantially causing any unwanted side effects or further addictive behaviors. Combination of GHB and a GABA_{B} receptor agonist may relieve the untoward side effects of the GABA_{B} receptor agonist, e.g., the combination allows for a lower dose of the GABA_{B} receptor agonist and thus a reduction in the potential side effects, especially somnolence.

The therapeutic amount of GABA_{B} receptor agonists present in the pharmaceutical compositions useful in this invention can cover a wide dosage range. The dose to be administered daily is selected to suit the desired effect and includes but is not limited to, the symptom suppressing dose (SSD) described in U.S. Ser. No. 12/014,746. In one embodiment, the amount of GABA_{B} receptor agonist administered daily per subject ranges from about 0.01 mg/kg body weight to about 200 mg/kg body weight. In another embodiment, the amount of GABA_{B} receptor agonist administered daily per subject ranges from about 0.05 mg/kg body weight to about 100 mg/kg body weight. If required, higher or lower daily doses can also be administered as can readily be determined by those of skill in the art.

The therapeutic amount of GHB present in the pharmaceutical compositions useful in this invention, which is to be administered, can cover a wide range. The dose to be administered daily is selected to suit the desired effect. In one embodiment, the amount of GHB administered daily per subject ranges from about 0.01 mg/kg body weight to about 200 mg/kg body weight. In another embodiment, the amount of GHB administered daily per subject ranges from about 0.05 mg/kg body weight to about 100 mg/kg body weight. If required, higher or lower daily doses can also be administered as can readily be determined by those of skill in the art.

The dosage levels of the GABA_{B} receptor agonists and GHB in the pharmaceutical compositions of the invention may be varied so as to obtain an amount which is effective to achieve the desired therapeutic response for a particular subject, composition, and mode of administration without being toxic to the subject. The selected dosage level will depend upon a variety of factors including the route of administration, the time of administration, the rate of excretion of the particular compound being employed, the duration of the treatment, other drugs, compounds and/or materials used in combination with the particular compounds employed, the age, sex, weight, condition, general health and prior medical history of the subject being treated, and like factors well known in the medical arts. Multiple administrations of the pharmaceutical compositions, each administration containing a fraction of the total daily desired amount, can also be given to achieve the desired therapeutic response for a particular subject.

The pharmaceutical compositions of the invention is made by methods known to those skilled in the art. Some simple techniques include, but not limited to, solid powder mixing, combining solutions and drying, co-precipitation method, and the like. Any method that produces a uniform mixture of powders can be suitably used for the preparation of the pharmaceutical compositions of the invention, and are well-known to one of ordinary skill in the art. Dosage forms can be compounded for individuals once the appropriate level has been determined for a given individual being treated for a particular addiction.

The pharmaceutical compositions of the invention can be administered orally, for example in the form of pills, tablets, coated tablets, capsules, granules or elixirs. Administration can also be carried out rectally, for example in the form of suppositories, or parentally, for example intravenously, intramuscularly, intrathecally or subcutaneously, in the form of injectable sterile solutions or suspensions, or topically, for example in the form of solutions or transdermal patches, or in other ways, for example in the form of aerosols or nasal sprays. Depending on the nature of the administration, the pharmaceutical compositions may further comprise, for example, pharmaceutically acceptable additives, excipients, carriers, and the like, that may improve, for example, manufacturability, administration, taste, ingestion, uptake, and so on.

The term “excipients” refer to pharmacologically inert ingredients that are not active in the body. See HANDBOOK OF PHARMACEUTICAL EXCIPIENTS (Am. Pharm. Ass'n 1986). One of ordinary skill in the art will recognize that many different excipients can be used in formulations according to the present invention and the list provided herein is not exhaustive.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

The pharmaceutical compositions of the invention may also contain adjuvants such as, but not limited to, preservatives, wetting agents, emulsifying agents, and dispersing agents. For example, prevention of the action of microorganisms may be achieved by the inclusion of various antibacterial and/or antifungal agents, for example, paraben, chlorobutanol, phenol sorbic acid, and the like. It also may be desirable to include isotonic agents, such as sugars, sodium chloride, and the like in the compositions. In addition, prolonged absorption of the injectable pharmaceutical form may be brought about by the inclusion of agents that delay absorption, such as aluminum monostearate and/or gelatin.

Pharmaceutically acceptable inert inorganic and/or organic carriers and/or additives that can be used: (a) for the production of pills, tablets, coated tablets and hard gelatin capsules include, for example, lactose, corn starch or derivatives thereof, gum arabic, magnesia or glucose, etc.; (b) for soft gelatin capsules and suppositories are, for example, fats, wax, natural or hardened oils, etc.; (c) for the production of solutions, for example injection solutions, or of emulsions or syrups are, for example, water, physiological sodium chloride solution or alcohols, for example, ethanol, propanol or glycerol, sugar solutions, such as glucose solutions or mannitol solutions, or a mixture of the various aforementioned solvents.

For oral administration, the pharmaceutical compositions of the invention can also be formulated into a liquid dosage form. Suitable formulations include emulsions, microemulsions, solutions, suspensions, syrups, and elixirs. These formulations optionally include diluents commonly used in the art, such as, for example, water or other solvents, solubilizing agents and emulsifiers, including, but not limited to, ethyl alcohol, isopropyl alcohol, ethyl carbonate, ethyl acetate, benzyl alcohol, benzyl benzoate, propylene glycol, 1,3-butylene glycol, oils, glycerol, tetrahydrofuryl alcohol, polyethylene glycols, fatty acid esters of sorbitan, and mixtures thereof. In addition, the liquid formulations optionally include adjuvants such as wetting agents, emulsifying and suspending agents, sweetening, flavoring, coloring, perfuming, and preservative agents. Suitable suspension agents include, but are not limited to, ethoxylated isostearyl alcohols, polyoxyethylene sorbitol and sorbitan esters, microcrystalline cellulose, aluminum metahydroxide, bentonite, agar-agar and tragacanth, and mixtures thereof. The liquid formulations may be delivered as-is, or may be provided in hard or soft capsules, for example.

As used herein, the term “intra-nasal” administration is meant to encompass those modes of administering a compound to a subject by means of absorption through the mucous membranes of the nasal cavity, or any administration that is made through the nasal cavity.

As used herein, the terms “buccal administration” and “sublingual administration” are meant to encompass those modes of administering a compound to a subject by means of absorption through the mucous membranes of the oral cavity, or any administration that is made where the drug is absorbed from the mouth. The pharmaceutical compositions can be adapted for buccal or sublingual administration by formulating them in the form of a tablet, patch, troche, or in free form, such as a gel, ointment, cream, or gum. Such formulations can also use a suitable adhesive to maintain the device in contact with the buccal mucosa. Examples of suitable adhesives are found in, for example, U.S. Pat. No. 4,948,580. The adhesive can comprise a matrix of a hydrophilic, e.g., water-soluble or -swellable, polymer or mixture of polymers that can adhere to a wet, mucous surface.

For parenteral administration, such as administration by injection (including, but not limited to, subcutaneous, bolus injection, intramuscular, intraperitoneal, intrathecal and intravenous), the pharmaceutical compositions may be formulated as isotonic suspensions, solutions, or emulsions, in oily or aqueous vehicles, and may contain formulatory agents such as suspending, stabilizing, and/or dispersing agents. Alternatively, the compositions may be provided in dry form such as a powder, crystalline, or freeze-dried solid, for reconstitution with sterile pyrogen-free water or isotonic saline before use. They may be presented, for example, in sterile ampoules or vials.

For rectal or vaginal administration, the pharmaceutical compositions of the invention can be provided as a suppository. Suppositories can comprise one or more non-irritating excipients such as, for example, polyethylene glycol, a suppository wax, or a salicylate. Such excipients can be selected on the basis of desirable physical properties. For example, a compound that is solid at room temperature but liquid at body temperature will melt in the rectal or vaginal cavity and release the active compounds. The pharmaceutical compositions can alternatively be provided as an enema for rectal delivery. Pharmaceutical compositions suitable for vaginal administration also include pessaries, tampons, creams, gels, pastes, foams, or spray formulations containing such carriers, examples of which are known in the art.

As used herein, the term “transdermal administration” is meant to encompass those modes of administering a compound to a subject by means of absorption through the skin. Pharmaceutical compositions can be suitably adapted for topical or transdermal administration by formulating them in the form of, but not limited to, powders, sprays, ointments, pastes, creams, lotions, gels, solutions, patches, and inhalants. Such formulations can contain excipients such as animal and vegetable fats, oils, waxes, paraffins, starch, tragacanth, cellulose derivatives, polyethylene glycols, silicones, bentonites, silicic acid, talc, zinc oxide, or mixtures thereof. Powders and sprays can also contain excipients such as lactose, talc, silicic acid, aluminum hydroxide, calcium silicates, and polyamide powder. Additionally, sprays can contain propellants, such as chlorofluoro-hydrocarbons and volatile unsubstituted hydrocarbons, such as butane and/or propane.

In one embodiment, the present invention provides a method for treatment of addictive disorders in a subject. The method involves the administration of the aforementioned pharmaceutical compositions of the invention, which comprises a therapeutic amount of at least one GABA_{B} receptor agonists and a therapeutic amount of GHB. IN another embodiment of the invention, the therapeutic amounts of GHB and the at least one GABA_{B} receptor agonist are administered as separate unit dosage forms. In both cases, the route of administration of the pharmaceutical composition(s) depends upon a variety of factors including the activity of the individual compounds or suitable derivatives thereof in the pharmaceutical compositions employed, the time of administration, the rate of excretion, the duration of the treatment, other drugs, compounds and/or materials used, the age, sex, weight, condition, general health and prior medical history of the subject being treated, and like factors well known in the medical arts.

The administration of the pharmaceutical compositions may be effected orally, intra-nasally, buccally, sublingually, transdermally, parenterally, intraventricular, or intrathecally. The pharmaceutical compositions can be formulated accordingly to suit the requirements of the chosen route of administration.

Accordingly, the invention provides a method of treatment of subjects suffering from addictive disorders. The method comprises administering a therapeutic amount of at least one GABA_{B} receptor agonist, and administering a therapeutic amount of GHB. The order of administration of the two compounds is not specific. Hence, in one embodiment, the therapeutic amount of at least one GABA_{B} receptor agonist is administered first, followed by the administration of therapeutic amount of GHB. In another embodiment, the therapeutic amount of GHB is administered first, followed by the administration of the therapeutic amount of at least one GABA_{B} receptor agonist.

The at least one GABA_{B} receptor agonist useful in the method include those mentioned previously, including optically active, racemic mixtures and enantiomerically pure forms of the compounds, derivatives, prodrugs metabolites and any pharmaceutically acceptable salts of the aforementioned compounds. In one specific embodiment, the at least one GABA_{B} receptor agonist useful in the invention is 4-amino-3-(4-chlorophenyl)butanoic acid, also known as baclofen. Also included herein are racemic baclofen, enantiomerically pure L-baclofen, and analogs, derivatives, prodrugs, metabolites thereof. For example, baclofen analogs include 3-thienyl- and 3-furylaminobutyric acids.

The therapeutic amount of baclofen to be administered can cover a wide range. The dose to be administered daily is to be selected to suit the desired effect and includes the SSD as described in U.S. Ser. No. 12/014,746 and as can be determined therein. In one embodiment, the amount of baclofen administered daily per subject ranges from about 0.01 mg/kg of body weight to about 200 mg/kg of body weight. In another embodiment, the amount of baclofen administered daily per subject ranges from about 0.05 mg/kg of body weight to about 50 mg/kg of body weight. If required, higher or lower daily doses can also be administered.

The at least one GABA_{B} receptor agonist useful in the invention can be administered orally, for example in the form of pills, tablets, coated tablets, capsules, granules or elixirs. Administration can also be carried out rectally, for example in the form of suppositories, or parentally, for example intravenously, intramuscularly, intrathecally or subcutaneously, in the form of injectable sterile solutions or suspensions, or topically, for example in the form of solutions or transdermal patches, or in other ways, for example in the form of aerosols or nasal sprays. Depending on the nature of the administration, the at least one GABA_{B} receptor agonist may be formulated with, for example, pharmaceutically acceptable additives, excipients, carriers, and the like, that may improve, for example, manufacturability, administration, taste, ingestion, uptake, and so on. Further, formulations of the at least one GABA_{B} receptor agonists may also contain adjuvants such as, but not limited to, preservatives, wetting agents, emulsifying agents, antibacterial/antimicrobial agents and dispersing agents.

The method also involves administration of a therapeutic amount of GHB. As noted, GHB can be employed as the free acid or alcohol, or as a pharmaceutically acceptable salt or ester thereof. Pharmaceutically acceptable salts may be obtained using standard procedures well known in the art, for example by reacting a sufficiently basic compound with the carboxylic acid affording a physiologically acceptable salt. Metal or organic cations, such as Ca⁺, K⁺, Li⁺, or (R)₄N⁺ wherein each R is H, phenyl, C₁-C₆ alkyl or hydroxyl C₁-C₆ alkyl, e.g., ammonium or hydroxyethyl amine salts of the carboxylic acid can be easily made by this method. Hydroxy protecting groups such as esters, ethers, acetals and ketals may be utilized in the present compounds. Useful hydroxy protecting groups are described in Greene et al., 1991.

The therapeutic amount of GHB to be administered can cover a wide range. The dose to be administered daily is to be selected to suit the desired effect. In one embodiment, the amount of GHB administered daily per subject ranges from about 0.01 mg/kg of body weight to about 200 mg/kg of body weight. In another embodiment, the amount of GHB administered daily per subject ranges from about 0.05 mg/kg body weight to about 50 mg/kg body weight. If required, higher or lower daily doses can also be administered.

The GHB useful in the invention can be administered orally, for example in the form of pills, tablets, coated tablets, capsules, granules or elixirs. Administration can also be carried out rectally, for example in the form of suppositories, or parentally, for example intravenously, intramuscularly, intrathecally or subcutaneously, in the form of injectable sterile solutions or suspensions, or topically, for example in the form of solutions or transdermal patches, or in other ways, for example in the form of aerosols or nasal sprays. Depending on the nature of the administration, the GHB may be formulated with, for example, pharmaceutically acceptable additives, excipients, carriers, and the like, that may improve, for example, manufacturability, administration, taste, ingestion, uptake, and so on. Further, formulations of GHB may also contain adjuvants such as, but not limited to, preservatives, wetting agents, emulsifying agents, antibacterial/antimicrobial agents and dispersing agents.

Actual dosage levels of the at least one GABA_{B} receptor agonist and GHB may be varied so as to obtain an amount which is effective to achieve the desired therapeutic response for a particular subject, composition, and mode of administration without being toxic to the subject. The selected dosage level will depend upon a variety of factors including the route of administration, the time of administration, the rate of excretion of the particular compound being employed, the duration of the treatment, other drugs, compounds and/or materials used in combination with the particular compounds employed, the age, sex, weight, condition, general health and prior medical history of the subject being treated, and like factors well known in the medical arts. Multiple administrations of the pharmaceutical compositions, each administration containing a fraction of the total daily desired amount, can also be given to achieve the desired therapeutic response for a particular subject.

The method of treatment of subjects suppresses or at least substantially reduces cravings associated with addictive disorders, without substantially causing any unwanted side effects or further addictive behaviors.

It will be appreciated by those skilled in the art that various omissions, additions and modifications may be made to the invention described above without departing from the scope of the invention, and all such modifications and changes are intended to fall within the scope of the invention, as defined by the appended claims. All references patents, patent applications or other documents cited are herein incorporated by reference in their entirety.

### REFERENCES

Addolorato, G., Caputo, F., Capristo, E. et al. (2002) Baclofen efficacy in reducing alcohol craving and intake: a preliminary doubleblind randomized controlled study. Alcohol Alcoholism 37, 504-508.

Agabio R, Marras P, Addolorato G, Carpiniello B, Gessa G L. (2007) Baclofen suppresses alcohol intake and craving for alcohol in a schizophreniac alcohol-dependent patient: a case report. J. Clin. Psychopharmacol. 27: 319-20.

Ameisen O. (2005) Complete and prolonged suppression of symptoms and consequences of alcohol-dependence using high-dose baclofen: a self-case report of a physician. (2005) Alcohol Alcoholism 40: 147-50.

Ameisen O. (2007a) Baclofen as a craving-suppressing agent. CNS Drugs 21:693.

Ameisen O. (2007b) Gamma-Hydroxybutyrate (GHB)-Deficiency in Alcohol-Dependence? Alcohol Alcoholism Adv. Access, Aug. 1, 2007.

Ameisen, O. (2008) Are the effects of gamma-hydroxybutyrate (GHB) treatment partly physiological in alcohol dependence? Am. J. Drug Alcohol Abuse 34:235-6; author reply 237-8.

Breslow, M. F., Fankhauser, M. P., Potter, R. L. et al. (1989) Role of gamma-aminobutyric acid in antipanic drug efficacy. Am. J. Psychiatry 146, 353-356.

Bucknam, W. (2007) Suppression of symptoms of alcohol dependence and craving using high-dose baclofen. Alcohol Alcoholism 42, 158-160.

Caputo F, Addolorato G, Stoppo M, Francini S, Vignoli T, Lorenzini F, Del Re A, Comaschi C, Andreone P, Trevisani F, Bernardi M. (2007) Comparing and combining gamma-hydroxybutyric acid (GHB) and naltrexone in maintaining abstinence from alcohol: An open randomised comparative study. Eur. Neuropsychopharmacol. 17:781-789.

Caputo, F., Addolorato, G. and Lorenzini, F. (2003) Gammahydroxybutyric acid versus naltrexone in maintaining alcohol abstinence: an open randomized comparative study. Drug Alcohol Depend. 70, 85-91.

Carai, M. A., Colombo, G., Brunetti, G. et al. (2001) Role of GABA(B) receptors in the sedative/hypnotic effect of gammahydroxybutyric acid. Eur. J. Pharmacol. 428, 315-321.

Davidson D, Wirtz P W, Gulliver S B, Longabaugh R. (2007) Naltrexone's suppressant effects on drinking are limited to the first 3 months of treatment. Psychopharmacology (Berl). 194:1-10 [Epub 2007 May 19].

Drake, R. G., Davis, L. L., Cates, M. E. et al. (2003) Baclofen treatment for chronic posttraumatic stress disorder. Ann. Pharmacother. 37, 1177-1181.

Greene, T. W.; Wutz, P. G. M., “Protecting Groups in Organic Synthesis”, 2nd ed., John Wiley & Sons, Inc (1991).

Humeniuk, R. E., White, J. M. and Ong, J. (1994) The effects of GABA_{B} ligands on alcohol withdrawal in mice. Pharmacol. Biochem. Behav. 49, 561-566.

Krupitsky, E. M., Burakov, A. M., Ivanov, V. B. et al. (1993) Baclofen administration for the treatment of affective disorders in alcoholic patients. Drug Alcohol Depend. 33, 157-163.

Nava, F., Premi, S., Manzato, E. and Lucchini, A. (2006) Comparing treatments of alcoholism on craving and biochemical measures of alcohol consumption. J. Psychoactive Drugs 38, 211-217.

Schweitzer, P., Roberto, M. and Madamba, S. G. (2004) Gammahydroxybutyrate increases a potassium current and decreases the H-current in hippocampal neurons via GABA_{B} receptors. J. Pharmacol. Exp. Thera. 311, 172-179.

I claim:

1. A pharmaceutical composition comprising therapeutically effective amounts of gamma-hydroxybutyrate (GHB) and at least one GABA_{B} receptor agonist sufficient to suppress cravings associated with an addiction.

2. The pharmaceutical composition of claim 1 wherein said GABA_{B} receptor agonist is baclofen.

3. The pharmaceutical composition of claim 1 wherein the therapeutic amount of the at least one GABA_{B} receptor agonist ranges from about 0.01 mg/kg of body weight to about 200 mg/kg of body weight.

4. The pharmaceutical composition of claim 1, wherein the therapeutic amount of GHB ranges from about 0.01 mg/kg of body weight to about 200 mg/kg of body weight.

5. The pharmaceutical composition of claim 1, formulated to be administered orally, intra-nasally, buccally, sublingually, transdermally, parenterally, intraventricular, or intrathecally.

6. The pharmaceutical composition of claim 1, wherein said addiction is to alcohol, nicotine, cocaine, heroin, another drug, gambling, sex, bulimia, binge-eating disorders or an obsessive-compulsive disorder.

7. A method of treating addiction which comprises co-administering therapeutically effective amounts of gamma-hydroxybutyrate (GHB) and at least one GABA_{B} receptor agonist sufficient to suppress cravings associated with an addiction.

8. The method of claim 7, wherein said GABA_{B} receptor agonist is baclofen.

9. The method of claim 7, wherein the therapeutic amount of the at least one GABA_{B} receptor agonist ranges from about 0.01 mg/kg of body weight to about 200 mg/kg of body weight daily.

10. The method of claim 7, wherein the therapeutic amount of GHB ranges from about 0.01 mg/kg of body weight to about 200 mg/kg of body weight daily.

11. The method of claim 7, wherein said addiction is to alcohol, nicotine, cocaine, heroin, another drug, gambling, sex, bulimia, binge-eating disorders or an obsessive-compulsive disorder.

12. The method of claim 7 wherein said GHB and said at least one GABA_{B} receptor agonist are administered in a single dosage form.

13. A method of treating addiction which comprises sequentially administering gamma-hydroxybutyrate (GHB) and at least one GABA_{B} receptor agonist for a time and in amounts sufficient to suppress cravings associated with said addiction

14. The method of claim 13, wherein the at least one GABA_{B} receptor agonist is administered first.

15. The method of claim 13, wherein the GHB is administered first.

16. The method of claim 13, wherein said GABA_{B} receptor agonist is baclofen.

17. The method of claim 13, wherein said addiction is to alcohol, nicotine, cocaine, heroin, another drug, gambling, sex, bulimia, binge-eating disorders or an obsessive-compulsive disorder.

18. A method to treat or ameliorate depression or dysfunctional psychological condition which comprises administering the pharmaceutical composition of claim 1 to a patient in need of treatment for a time and in an amount to relieve the associated symptoms of the depression or the condition and to provide therapeutic benefit to the patient.

19. The method of claim 18, wherein said condition is menopause, premenstrual symptoms, insomnia, obsessive-compulsive disorder, ADHD, ADD or tensions headaches.
